(19) United States
(12) Patent Application Publication
(10) Pub. No.: US 2014/0249401 A1
(43) Pub. Date: Sep. 4, 2014

(54) APPARATUS AND METHOD FOR MR EXAMINATION, AND TEMPERATURE CONTROL SYSTEM AND METHOD

(71) Applicant: KONINKLIJKE PHILIPS N.V., EINDHOVEN (NL)

(72) Inventors: Johan Samuel Van Den Brink, Eindhoven (NL); Paul Royston Harvey, Eindhoven (NL); Peter Forthmann, Hamburg (DE); Christoph Leussler, Hamburg (DE); Peter Vernickel, Hamburg (DE); Jan Hendrik Wülbern, Hamburg (DE); Ingmar Graesslin, Hamburg (DE)

(73) Assignee: KONINKLIJKE PHILIPS N.V., EINDHOVEN (NL)

(21) Appl. No.: 14/350,830

(22) PCT Filed: Oct. 3, 2012
(86) PCT No.: PCT/IB2012/055294

§ 371 Date: Apr. 10, 2014

Related U.S. Application Data
(60) Provisional application No. 61/545,627, filed on Oct. 11, 2011.

(51) Int. Cl. G01R 33/31; G01R 33/44; A61B 5/055

(52) U.S. Cl. CPC G01R 33/31; A61B 5/055; G01R 33/44
USPC 600/410

(57) ABSTRACT

The invention provides an apparatus (1) for magnetic resonance (MR) examination of a subject (S), comprising: an examination region (3) for accommodating the subject (S) during the MR examination; a radio-frequency system (5) for transmission of a radio-frequency (RF) signal or field into the examination region (3) during the MR examination; and a temperature control system (6) for controlling the temperature of the subject (S) in the examination region (3) during the examination. The temperature control system (6) is configured to actively control or regulate an environment of the subject (S), and thereby the temperature or thermal comformt of the subject (S) based upon a detected and/or an expected temperature of the subject (S) during the MR examination. The invention also provides a method of controlling thermal comfort of the subject (S) during an examination of the subject (S) in a MR apparatus (1), comprising the steps of: estimating and/or detecting a temperature of the subject (S) during the MR examination, and actively controlling or regulating the environment of the subject (S) based upon the estimated and/or detected temperature of the subject (S) during the MR examination.

### FIELD OF THE INVENTION

The present invention relates to the field of magnetic resonance (MR), and more particularly to an apparatus and method for magnetic resonance (MR) examination of a subject, especially a human, as well as to a temperature control system and method for a MR apparatus and to a computer program product for implementing the method of the invention.

### BACKGROUND OF THE INVENTION

Magnetic resonance (MR) apparatus, and particularly magnetic resonance imaging (MRI) apparatus, have become important tools in the examination of individuals for the analysis and assessment of a whole range of different conditions. One consequence of using MR apparatus, however, is that the subject is typically exposed to significant amounts of radio-frequency (RF) energy during an examination. The specific absorption rate (SAR) of RF energy by individuals who undergo examination with a MR apparatus is therefore the subject of strict regulation in various countries.

Nevertheless, some MRI scan protocols still impart substantial amounts of RF energy to the body of the individual examined. Because the individual is often surrounded or enclosed by coil arrays on a bed or table during the MR examination, these can act as thermal insulators and impair the dissipation of any heat generated. As an example, while the metabolic heat generation of a person is typically about 100 W, the RF energy imparted to an individual during examination with a conventional MRI apparatus can easily be in the range of 200 to 300 W. As such, this RF energy creates an additional heat load for the individual and can lead to sweating, discomfort, an increase in body core temperature, and may also impose restrictions on the MRI scan protocols to be applied.

Some attempts have been made to address the heating of the subject during an MR examination. For instance, US Patent Application Publication No. US 2010/0253338 A1 is directed to the issue of SAR in individuals having metallic implants, such as prostheses and pacemakers. In that case, a particular design of the RF coil was combined with controlling or tailoring the RF signal transmission to minimize the RF heating of the metallic implant. That system does not, however, address the more general issue of sweating and discomfort in the absence of an implant. While the provision of an air-conditioned clinical treatment room for conducting an MR examination is known, this in itself does not address the issue of localized heating and discomfort of the individual during an MR examination. Accordingly, a need remains for improvements in addressing the heating issues discussed above and enhancing the comfort of MR examination.

### SUMMARY OF THE INVENTION

It is thus an object of the present invention to provide a new and improved apparatus and method for magnetic resonance (MR) examination of a subject (e.g. a human), which address the disadvantages and limitations of known MR apparatus discussed above.

To this end, the invention provides an apparatus for magnetic resonance examination of a subject as recited in claim 1 and method of magnetic resonance examination of a subject as recited in claim 8. Furthermore, the invention provides a method of controlling an environment of a subject, and thereby thermal comfort or temperature of the subject, in a magnetic resonance examination as recited in claim 9, as well as a computer program product as recited in claim 14. Preferred features are recited in the dependent claims.

According to one aspect, therefore, an apparatus for magnetic resonance (MR) examination of a subject is provided, comprising: an examination region for accommodating the subject during the MR examination, a radio-frequency system for transmission of a radio-frequency (RF) signal or field into the examination region during the MR examination, and a temperature control system for controlling an environment of the subject and thereby thermal comfort of the subject in the examination region during the examination. The temperature control system is configured to actively control or regulate the environment of the subject based upon a detected and/or an expected temperature of the subject during the MR examination. Desirably, the temperature control system is configured to actively control or regulate the environment and thereby the thermal comfort or temperature of the subject throughout or over a duration of the MR examination.

In a preferred embodiment, the temperature control system includes: at least one cooling device for generating a cooling effect on the subject accommodated in the examination region, and a control unit configured to adjust operation of the at least one cooling device, and thereby to adjust the cooling effect, during the MR examination based upon the detected and/or expected temperature of the subject during the MR examination. The at least one cooling device is preferably adapted to generate the cooling effect on the subject via a heat transfer medium in thermal contact with the subject. As such, the control unit is preferably configured to modify or adjust a temperature and/or a flow rate of the heat transfer medium based on the detected and/or the expected temperature of the subject during the MR examination.

With existing MR apparatus, the ambient temperature in the examination room is often set very low to counteract any heating of the human subject or individual during the examination, which consequently causes the individual to feel cold and uncomfortable, and often leads to blankets being provided. Blankets, on the other hand, not only complicate the work-flow of the technicians carrying out the MR examination, but are counter-productive when heating and sweating is to be prevented during the examination. The present invention is thus able to provide improved temperature control during the MR examination. At the start of examination, for example, the temperature of the individual should be comfortable, with little cooling required. During the examination, however, the RF energy imparted to the individual increases. As such, the temperature control system is able to adjust operation of the at least one cooling device and thereby adapt the cooling effect to the heating experienced by the individual. This way it is possible to prevent the subject feeling cold, especially at the start of an examination, and the undesirable sensations of being too warm or sweating during the examination are also avoided. The work-flow for the technicians conducting the MR examinations can also be improved, as no blankets need to be provided or removed.

In a preferred embodiment, the at least one cooling device comprises an air-flow generator, such as a fan or ventilator, for generating an air-flow over the subject in the examination region during the MR examination. The air-flow generator may be arranged in the apparatus in variety of locations to direct the flow of air at the subject. For example, the air-flow generator may be located to generate (a) air flow through a table or support on which the subject is positioned; (b) air flow from a side or lower part of the examination region; or (c) air flow from a coil of the apparatus, e.g. over or around the subject's head, which has a very large cooling capacity, and may greatly influence comfort sensations. Thus, the control unit may actively regulate or control a temperature and/or a flow rate of the air from the air-flow generator to adjust the cooling effect generated by the air-flow. Notably, the air-flow is directed to cool the subject or individual who is exposed to heat energy from the SAR deposited by the transmitted RF fields of the magnetic resonance examination apparatus. As discussed in more detail below, the air-flow may be adjusted based on the expected SAR for the current MR signal acquisition sequence or on actual SAR detected from the subject.

In a preferred embodiment, the at least one cooling device comprises a cooling pad containing a heat transfer medium. The heat transfer medium is preferably a liquid, such as an oil, water, D₂O or a gel, and the cooling pad may be designed such that the heat transfer liquid flows through the pad, e.g. in a continuous flow from an inlet to an outlet of the pad. As such, the heat transfer liquid may be supplied from a reservoir and conveyed through the cooling pad by known means, such as a pump. Thus, the control unit may actively regulate or control a temperature and/or a flow rate of the heat-transfer liquid in the cooling pad to adjust the cooling effect generated by the pad.

Desirably, the cooling pad is configured for contact with or application to the subject for a duration of the MR examination. The cooling pad should fit the body surface as well as possible in order to provide an optimum thermal conductivity interface. Accordingly, the cooling pad is preferably configured to at least partially wrap around, envelop or cover a part of the subject. This can be achieved by providing the cooling pad with a relatively flat flexible structure having a soft material composition, to be filled or inflated with the said heat transfer medium. An accessible and unobtrusive, yet still very effective place to apply the cooling pad is the calf or thigh region of an individual, which is often outside a field-of-view of the RF signal, and blood flow in the individual transports heat generated by the RF thermal load to the cooling pad. The cooling pad may alternatively be designed to fit the forearm, or the head (noted for its large cooling capacity, and influence on comfort sensations) or could be designed more universally to fit more than one body region. The cooling pad could also be incorporated in the examination region so that locating the subject in the examination region automatically places the subject in contact with the cooling pad. This would simplify the work-flow for the technicians conducting the examination also, as they would not need to apply the cooling pad to the subject. In this connection, the cooling pad could be integrated in an examination table upon which the subject lies, or could be integrated with the coil array around the subject.

In a preferred embodiment, the “expected” temperature of the subject during the MR examination may be predicted or estimated using a model or simulation of the MR examination, and more particularly a model or simulation of the of RF energy to be deposited during the examination and/or of the specific absorption rate (SAR) of the RF energy by the subject undergoing the examination (i.e. the global and/or local SAR). In this connection, it can be particularly useful to model local SAR, and especially in those regions where the most heating is expected, namely in the extremities and/or in the subject's skin. Alternatively, or in addition, the model may also evaluate a CEM43 value (i.e. Cumulative Equivalent Minutes at 43 Degrees Celsius) for the subject. Thus, in a preferred embodiment, the temperature control system is able to estimate a prospective RF thermal load for the subject generated throughout or over a duration of the examination and then actively control or regulate the temperature of the subject based on this estimation. In this regard, the temperature control system preferably includes software that performs an estimation to determine an expected RF thermal load and an associated temperature increase for the subject based upon information about the subject, such as size, weight, age, and/or thermo-regulatory capacity (e.g. clothing) of the subject, and can adjust operation of the cooling device(s) to adjust the cooling effect accordingly. Using a model of the imparted or deposited energy (per unit of time), and accounting for the subject's thermo-regulatory capacity (e.g. via a generic model of the subject or based on specific real data inputs), the required cooling capacity can be estimated and adjusted accordingly. Alternatively or in addition, as explained in more detail below, the actual RF energy imparted to the subject can be ascertained by detecting and monitoring the temperature of the subject throughout the MR examination, and the cooling capacity can be adjusted accordingly.

In a preferred embodiment, the temperature control system includes at least one detector for detecting the temperature of the subject during the MR examination, and particularly those parts of the subject in or adjacent to the examination region. As such, the “detected” temperature of the subject during the MR examination is the temperature detected or ascertained by the detector(s). Where more than one detector detects or ascertains more that one temperature at any given time, the highest of those values may be considered to be the most crucial or decisive. The detector may comprise a thermometry scan employing MRI for temperature mapping of RF heating during the MR examination. Preferably, the detector includes at least one temperature sensor, e.g. a MRI compatible, local temperature sensor. Each temperature sensor may be a separate device configured for application to the subject or individual in the examination region or may itself be integrated in the examination region; e.g. in a surface of the support table or in the coil array. Again, it can be particularly useful to monitor or detect the temperature of the subject in those regions where the most heating is expected, namely at the extremities and/or at a surface of the subjects's skin.

The temperature control system, and particularly the control unit, is desirably configured to adjust operation of the at least one cooling device to increase the cooling effect it generates if the detected temperature of the subject exceeds one or more predetermined threshold value. In this regard, the temperature control system may have a series of predetermined threshold values for the detected temperature, e.g. at 1° C. intervals, each of which causes a respective adjustment to the operation of the cooling device(s). The temperature control system, and particularly the control unit, is preferably also configured to adjust operation of the cooling device(s) to correspondingly reduce the cooling effect generated if the detected temperature of the subject falls below one or more predetermined threshold values. Thus, an automatic measurement of local surface temperature and temperature gradients provides input for a regulation system using a feedback loop. That is, the detected or expected temperature of the subject is periodically sampled over a duration of the MR examination and the at least one cooling device is correspondingly adjusted by the control unit periodically. Preferably, the control unit provides programmable and pre-set variable gradient temperature selection—e.g. in both a cooling and a heating mode—which allows either rapid or gradual subject temperature management. This enables active and more accurate control of the temperature or thermal comfort of a person in the examination region of the apparatus within a pre-set range; for example in the range of 20 to 25° C., and more preferably in the range of 22 to 23° C. In this regard, it will be noted that the termperature at the surface of the skin of subject is typically highly relevant for the degree of thermal comfort experienced. The invention therefore both increases the comfort of the individual being examined and obviates cumbersome provision and removal of blankets.

According to another aspect, a temperature control system is provided for controlling an environment of a subject and thereby thermal comfort of the subject during a magnetic resonance examination, wherein the temperature control system is configured to actively control or regulate the environment and thereby the temperature of the subject based upon a detected and/or an expected temperature of the subject during the MR examination. The temperature control system is preferably configured to actively control or regulate the environment and thereby the temperature of the subject throughout or over a duration of the MR examination. Furthermore, the various features of the temperature control system in the preferred embodiments discussed above apply equally in this aspect and are not repeated here for the sake of economy. A stand-alone temperature control system may therefore come as a wearable, e.g. wrap-around, optionally disposable, cooling pad system.

According to a further aspect, a method of magnetic resonance examination of a subject is provided, comprising the steps of: accommodating the subject in an examination region of a magnetic resonance apparatus for MR examination of the subject; transmitting a radio-frequency (RF) signal or field into the examination region during the MR examination; estimating and/or detecting a temperature of the subject during the MR examination; and actively controlling or regulating an environment of the subject and thereby thermal comfort of the subject based upon the estimated and/or detected temperature of the subject during the MR examination.

According to yet another aspect, a method is provided for controlling an environment and thereby a temperature or thermal comfort of a subject during a magnetic resonance (MR) examination of the subject in a MR apparatus having an examination region for accommodating the subject and a radio-frequency system for transmitting a radio-frequency (RF) signal or field into the examination region during the MR examination, the method comprising the steps of: estimating and/or detecting a temperature of the subject during the MR examination; and actively controlling or regulating the environment and thereby the temperature or thermal comfort of the subject based on the estimated and/or detected temperature of the subject during the MR examination.

As will be appreciated, the above methods do not involve any therapeutic or surgical treatment of the subject or individual undergoing the MR examination, nor are these methods diagnostic in nature. In particular, while a MR examination can acquire data that in some instances may contribute to an overall clinical picture of a subject, the MR examination itself does not have a diagnostic character. Further, it will be noted that not all of the steps of the above methods require interaction with the subject or individual.

In a preferred embodiment of the above methods, the step of estimating and/or detecting a temperature of the subject is performed repeatedly at intervals over a duration of the MR examination, and the step of actively controlling or regulating the environment of the subject is iterative and based upon the most recent estimated and/or detected temperature. In this manner, a succession of estimated and/or detected temperatures of the subject may be obtained over the duration of the MR examination, such that the active control or regulation of the environment of the subject is iterated or “updated” based on each newly estimated or detected temperature. Where an embodiment of the method and apparatus provides both an estimated temperature and a detected temperature of the subject for a specific point in time, the detected temperature will typically be treated as the decisive or critical temperature upon which the step of actively controlling or regulating the temperature of the subject is based. Preferably, however, the method of the invention involves a repeated comparison between the estimated and the detected temperatures to identify significant discrepancies or anomalies and so ensure a proper functioning of the apparatus performing the MR examination as well as the safety of the individual.

In a preferred embodiment, the MR apparatus includes at least one cooling device for generating a cooling effect on the subject accommodated in the examination region, and the step of actively controlling or regulating the environment of the subject comprises: adjusting operation of the at least one cooling device during the MR examination based upon the estimated and/or detected temperature of the subject during the examination.

In a preferred embodiment, the at least one cooling device is adapted to generate the cooling effect on the subject via a heat transfer medium, and the step of adjusting operation of the at least one cooling device comprises: adjusting a temperature and/or a flow rate of the heat transfer medium based upon the estimated and/or detected temperature of the subject during the MR examination.

In a preferred embodiment, the at least one cooling device comprises an air-flow generator for generating a flow of air over the subject in the examination region, wherein the step of adjusting operation of the at least one cooling device comprises: adjusting the flow rate of the air from the air-flow generator based upon the estimated and/or detected temperature of the subject during the MR examination.

In a preferred embodiment, the at least one cooling device comprises a cooling pad containing a heat transfer liquid, and the step of adjusting operation of the at least one cooling device comprises: adjusting a temperature and/or a flow rate of the heat transfer liquid in the cooling pad based upon the estimated and/or detected temperature of the subject during the MR examination. Preferably, the method comprises: applying the cooling pad to the subject for the MR examination, such that the cooling pad at least partially wraps around, envelops or covers part of the subject.

In a preferred embodiment, the step of estimating a temperature of the subject during the MR examination comprises: estimating a thermal load to be imparted to the subject using a model or simulation of the MR examination protocol, and more particularly a model or simulation of the of RF energy to be deposited during the examination and the specific absorption rate (SAR) of the energy by the subject undergoing the examination (i.e. global and/or local SAR). In this connection, it can be particularly useful to model local SAR, and especially in those regions where the most heating is expected, namely in the extremities and/or in the subject's skin. Alternatively, or in addition, the model may also evaluate a CEM43 value (i.e. Cumulative Equivalent Minutes at 43° C.) for the subject. By using a model of the imparted or deposited energy (per unit of time) and the individual's thermo-regulatory capacity (e.g. using a generic model for the individual or specific anatomical data), the required cooling capacity can be estimated and adjusted accordingly.

In a preferred embodiment, the step of detecting a temperature of the subject during the MR examination comprises at least one of: conducting a thermometry MR scan for temperature mapping of RF heating during the MR examination.

In a preferred embodiment, the step of detecting a temperature of the subject during the MR examination comprises: sensing the temperature of the subject (i.e. individual) via a temperature sensor applied to the subject and/or integrated in the examination region; e.g. in the surface of the bed or in the surface coils.

In a preferred embodiment, the step of adjusting operation of the at least one cooling device comprises: increasing the cooling effect when the detected temperature of the subject exceeds one or more predetermined threshold value. As noted above, a series of predetermined threshold values may be provided for the detected temperature, e.g. at specific intervals, each of which causes a respective adjustment to the operation of a cooling device.

According to still a further aspect, a computer program product is provided which may include or be provided on a computer-readable medium on which the program is stored. The computer program product is loadable into the internal memory of a digital computer and comprises software code portions for performing the method of controlling a temperature of a subject during a MR examination as described above when said computer program product is run on a computer.

With the temperature control system and method contemplated by the present invention, and particularly employing the active control or regulation of cooling devices as described above, the individual's thermo-regulation system is supported and his/her comfort can be considerably improved for high SAR MR scans. Consequently, larger global SAR values may also be allowable which, in turn, has the potential to increase MR image quality and/or to speed up the MR examination procedure.

### BRIEF DESCRIPTION OF THE DRAWINGS

These and other aspects of the invention will be apparent from and elucidated with reference to the embodiments described hereinafter. In the drawings:

FIG. 1 shows a schematic representation of an apparatus and method for MR examination of a subject according to a preferred embodiment; and

FIG. 2 shows a diagram schematically illustrating the inputs and outputs of a temperature control system for controlling a temperature of a subject during MR examination according to a preferred embodiment.

### DETAILED DESCRIPTION OF EMBODIMENTS

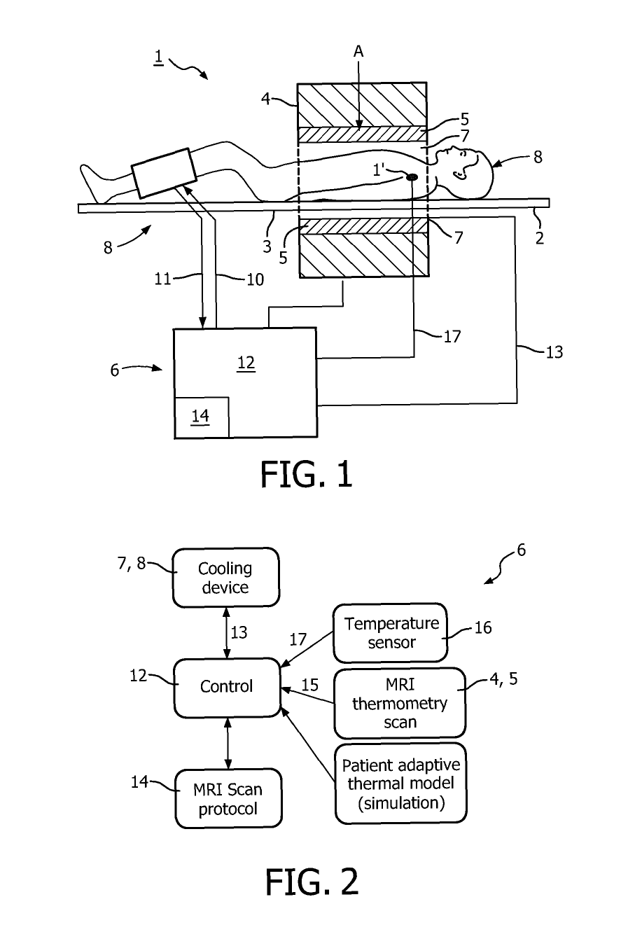

With reference firstly to FIG. 1 of the drawings, an ultra high field magnetic resonance imaging (MRI) apparatus 1 for magnetic resonance (MR) examination of subject S (in this case, a human subject or individual) according to a preferred embodiment is shown. The individual S is resting in a horizontal posture on a table 2 of the apparatus 1 with that part of the individual's body (i.e. torso) to be examined or imaged being accommodated in an examination region or space 3 formed by a cylindrical cavity formed in a housing 4 of the apparatus 1. As is known in the art, the MRI apparatus 1 includes one or more powerful magnet, gradient coils and a radio-frequency (RF) system 5 having an RF coil for transmission of a RF signal or field into the cylindrical examination region 3 in the direction of arrow A during the MR examination, all of these components forming a MR generation unit and being conveniently housed within the housing 4. The cylindrical cavity in the housing 4 which forms the examination region 3 is dimensioned such that the body of the individual or subject S is very close to the sides or walls of the cavity, thus providing only very limited access.

The MRI apparatus 1 of this embodiment further includes a temperature control system 6 for controlling an environment of the the subject S and thereby the temperature or the thermal comfort of the subject S in the examination region 3 over a duration of the MR examination. The temperature control system 6 includes two different cooling devices 7, 8 for cooling the individual S during the MR examination to counteract the thermal load imparted to the the individual's body in the examination region 3 by the RF signal or field from the radio-frequency system 5. The first cooling device 7 is a fan or ventilator having an annular configuration for generating a forced flow of air through the cylindrical cavity 3 around the individual S. As an alternative, however, the fan or ventilator 7 could direct the cooling air-flow over the subject's head outside the examination region 3. The second cooling device 8 comprises a cooling pad 9 in the form of flexible sleeve or cuff, which is designed to wrap around the calf or calves of the subject S. The pad-like sleeve or cuff 9 is filled with a liquid that acts as a heat-transfer medium. The second cooling device 8 further includes a supply line 10 for supplying the heat-transfer liquid to the sleeve or cuff 9 from a reservoir, and a return line 11 for returning the heat-transfer liquid from the cuff 9 to the reservoir.

Furthermore, the temperature control system 6 of the apparatus 1 comprises a control unit 12, including a computer processor, which is configured to periodically adjust operation of the two cooling device 7, 8 over a duration of the MR examination (thereby adjusting the cooling effect they generate on the body of the subject S) in dependence upon the thermal loading that the subject S is predicted or expected to receive from the RF field of the radio-frequency system 5 during the MR examination and in dependence upon the thermal loading that the individual is actually detected as having received from the radio-frequency system 5 during the MR examination. The control unit 12 is therefore operatively connected with the first cooling device 7 via a line 13 and is also operatively connected with the second cooling device 8 via the lines 10, 11 and a pump (not shown).

Stored in the processor memory 14 of the control unit 12 is a simulation or model for the RF energy projected into the examination region 3 and the specific absorption rate (SAR) of RF energy for each type of MR examination that the apparatus 1 performs. Furthermore, parameters that describe critical characteristics of the subject S, such as weight, age, and clothing, are able to be entered into the control unit 12 as input to be taken into account in the SAR simulation or model. Using the simulation or model for a particular MR examination, the control unit 12 is then able to estimate or predict the RF energy loading, and thus the heating or temperature, that the body of the subject S in the examination region 3 is expected to experience over the duration of the MR examination. In this way, even before an examination of the subject S in the apparatus 1 begins, the temperature control system 6 can determine or anticipate an expected heating or temperature of the subject S. This can be particularly helpful at the beginning of the examination, e.g. in the first minute.

The temperature control system 6 of the apparatus 1 also has two means for detecting the actual temperature of the body of the subject S within the examination region 3 during the course of the MR examination. One of these is via a thermometry MR scan to perform a temperature mapping of RF heating during the MR examination via the MRI components in the housing 4 of the apparatus 1, operatively connected to the control unit 12 via a line 15. The second means of detecting the actual temperature of the individual's body is via a temperature sensor 16, which is applied to the body of the individual S in the examination region 3 and is connected with the control unti 12 via a line 17. (It will be noted that each of the “lines” 13, 15, 17 operatively connecting to the control unit 12 simply denotes a channel of electro-magnetic connection. This may optionally be a physical wired or cable connection or alternatively it may be wireless). Thus, by virtue of the temperature detectors 4, 5, 15 and 16, 17, the temperature control system 6 is also able to detect an actual temperature of the body of the individual S in the cylindrical cavity 3 during the MR examination. By sampling the values or readings detected by the temperature detectors 4, 5, 15 and 16, 17 periodically, it is also possible to detect the actual temperature over the duration of the MR examination.

In the course of an MR examination, therefore, the control unit 12 of the control system 6 is configured to increase the operation of one or both of the first and second cooling devices 7, 8 when the expected temperature or the detected temperature of subject S exceeds a certain threshold value during the MR examination. The following scenario of operation of the temperature control system 6 is set out by way of example only and it will be understood that the specific cooling scheme with the cooling devices 7, 8 can be varied as deemed appropriate. Thus, for example: The annular fan or ventilator 7 might be switched off at the start of the MR examination, but if the expected temperature of the subject S after 30 seconds were to be, say, 1° C. above a certain threshold value, the fan 7 may be switched on by the control unit 12 after 30 seconds to iniate a gentle forced flow of air over the subject's body. Furthermore, if the expected temperature of the subject S after 1 minute were to be, say, 2° C. above a threshold value, the control unit 12 could adjust the fan 7 after 1 minute to increase the fan speed. Similarly, if the detected temperature of the subject S ascertained by the sensor 16 were, for example, to be 3° C. above a threshold value, the control unit 12 could operate to fill the cuff or sleeve 9 of the second cooling device 8 with cooling liquid to cool the individual's calves and optionally further increase the speed of the fan 7. If then, as a result of the increased cooling effect on the individual S by the fan 7 and the pad-like cuff 9, the detected temperature of the individual S were to reduce sufficiently—i.e. to below a threshold value—the control unit 12 could again adjust the operation of the first and second cooling devices 7, 8, but this time to reduce the cooling effect; e.g. by lowering or switching off the fan 7 and/or by stopping the flow of cooling liquid to the cuff 9.

In order to ensure a proper functioning of the temperature control system 6 as well as the safety of the individual S during MR examination, the expected temperature can be periodically compared with the detected temperature and, if significant discrepancies occur, the technicians who are conducting the examination can be automatically notified.

Referring now to FIG. 2 of the drawings, a schematic diagram of the operative components of the temperature control system 6 of the apparatus 1 and the connections, with and relationship to, the control unit 12 are illustrated. In FIG. 2, the same reference symbols have been used in respect of components corresponding to those described above in FIG. 1.

While the invention has been illustrated and described in detail in the drawings and foregoing description, such illustration and description are to be considered illustrative or exemplary only, and not restrictive. As such, the invention is not limited to the embodiments disclosed. Other variations to the disclosed embodiments can be understood and effected by those skilled in the art in practicing the claimed invention, from a study of the drawings, the disclosure, and the appended claims. In the claims, the word “comprising” does not exclude other features, elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### REFERENCE SYMBOL LIST

1 MRI apparatus
2 table support for a subject
3 MR examination region
4 housing of MR generation unit
5 radio-frequency system
6 temperature control system
7 first cooling device or fan
8 second cooling device
9 cooling pad as cuff or sleeve
10 supply line
11 return line
12 control unit
13 connecting line to fan
14 processor memory
15 connecting line to MR generation unit
16 temperature sensor
17 connecting line to temperature sensor
S subject or individual

1. An apparatus for magnetic resonance (MR) examination of a subject, comprising:
an examination region for accommodating the subject during the MR examination;
a radio-frequency system for transmission of a radio-frequency (RF) signal or field into the examination region during the MR examination;
a temperature control system for controlling thermal comfort of the subject t accommodated in the examination region during the examination,
wherein the temperature control system is configured to actively control or regulate the environment of the subject based upon a detected and/or expected temperature of the subject during the MR examination, and
wherein the temperature control system includes:
at least one cooling device for generating a cooling effect on the subject accommodated in the examination region, wherein the at least one cooling device comprises a cooling pad containing a heat transfer medium, wherein the cooling pad is configured for contact with, or application to, the subject for a duration of the MR examination, wherein the cooling pad is configures to at least partly wrap around. envelope or cover a part of the subject, and
a control unit configured to adjust operation of the at least one cooling device during the MR examination based upon the detected and/or expected temperature of the subject during the MR examination.

2. (canceled)

3. The apparatus of claim 2, wherein the temperature control system includes:
at least one detector for detecting the temperature of at least part of the subject in or adjacent to the examination region during the MR examination,
wherein the control unit is configured to adjust operation of the at least one cooling device to increase the cooling effect on the subject if the detected temperature of the subject exceeds one or more predetermined threshold value.

4. The apparatus of claim 2, wherein the at least one cooling device is configured to generate the cooling effect on the subject via the heat transfer medium in thermal contact with the subject, and wherein the control unit is configured to modify a temperature and/or a flow rate of the heat transfer medium based upon the detected and/or expected temperature of the subject during the MR examination.

5. (canceled)

6. The apparatus of claim 2, wherein the at least one cooling device comprises an air-flow generator for generating a flow of air over the subject accommodated in the examination region during the MR examination.

7. The apparatus claim 1, wherein the expected temperature of the subject during the MR examination is calculated as an estimation by a model or simulation of the specific absorption rate of the energy by the subject during the MR examination.

8. (canceled)

9. A method of controlling an environment of a subject for thermal comfort during a magnetic resonance examination of the subject in a MR apparatus having an examination region for accommodating the subject and a radio-frequency system for transmitting a radio-frequency signal or field into the examination region during the MR examination, the method comprising the steps of:
estimating and/or detecting a temperature of the subject during the MR examination, and
actively controlling or regulating the environment of the subject accommodated in the examination region based upon the estimated and/or detected temperature of the subject during the MR examination,
wherein the MR apparatus includes at least one cooling device for generating a cooling effect on the subject accommodated in the examination region, and wherein the step of actively controlling or regulating the environment of the subject comprises:
adjusting operation of the at least one cooling device to adjust the cooling effect during the MR examination based upon the estimated and/or detected temperature of the subject during the examination, wherein the at least one cooling device comprises a cooling, pad containing a heat transfer medium, wherein the cooling pad is configured for contact with, or application to, the subject for a duration of the MR examination, wherein the cooling pad is configured to at least partly wrap around, envelop or cover a part of the subject.

10. (canceled)

11. The method of claim 9, wherein the step of estimating and/or detecting a temperature of the subject is performed repeatedly at intervals over a duration of the MR examination, and wherein the step of actively controlling or regulating the environment of the subject is iterative and based upon the most recent estimated and/or detected temperature.

12. The method of claim 10, wherein the at least one cooling device is adapted to generate the cooling effect on the subject via a heat transfer medium, and wherein the step of adjusting operation of the at least one cooling device comprises:
adjusting a temperature and/or a flow rate of the heat transfer medium based upon the estimated and/or detected temperature of the subject during the MR examination.

13. The method of claim 9, wherein, when the step of estimating and/or detecting a temperature of the subject during the MR examination provides both an estimated temperature and a detected temperature of the subject for a specific point in time, the step of actively controlling or regulating the environment of the subject is based upon one or other of the estimated temperature and the detected temperature.

14. A computer program product loadable into the memory of a digital computer, comprising software code portions for performing the method of controlling a temperature of a subject during a MR examination according to claim 9 when said computer program product is run on a computer.
